(19) United States
(12) Patent Application Publication
(10) Pub. No.: US 2020/0126649 A1
(43) Pub. Date: Apr. 23, 2020

(54) DATA PROCESSING APPARATUS, DATA PROCESSING METHOD, AND DATA PROCESSING PROGRAM

(71) Applicants: OMRON HEALTHCARE CO., LTD., Muko-shi (JP); OMRON Corporation, Kyoto-shi (JP)

(72) Inventors: Hironori SATO, Kyoto (JP); Ayumu TAKAGI, Kyoto (JP); Daisuke NOZAKI, Kyoto (JP); Fumihiko NAKAMURA, Kyoto (JP)

(73) Assignees: OMRON HEALTHCARE CO., LTD., Muko-shi (JP); OMRON Corporation, Kyoto-shi (JP)

(21) Appl. No.: 16/718,467

(22) Filed: Dec. 18, 2019

Related U.S. Application Data
(63) Continuation of application No. PCT/JP2018/028820, filed on Aug. 1, 2018.

(30) Foreign Application Priority Data
Aug. 9, 2017 (JP) 2017-154740

(51) Int. Cl. G16H 15/00; G16H 10/60; G16H 20/10; G16H 50/30

(52) U.S. Cl. CPC G16H 15/00; G16H 50/30; G16H 20/10; G16H 10/60

(57) ABSTRACT

According to one embodiment, the present disclosure can provide a technique that supports examination practice on a patient by medical personnel. A data processing apparatus includes a biological data acquisition unit that acquires biological data of a subject, a remaining amount estimation unit that estimates a remaining amount of a medicine prescribed to the subject, a display data generating unit that generates display data that configures a display screen including a biological data display indicating the biological data and a remaining medicine amount display indicating the remaining amount, and a display data output unit that outputs the display data.

This application is a Continuation Application of PCT Application No. PCT/JP2018/028820, filed Aug. 1, 2018 and based upon and claiming the benefit of priority from prior Japanese Patent Application No. 2017-154740, filed Aug. 9, 2017, the entire contents of all of which are incorporated herein by reference.

### FIELD

The present invention relates generally to data processing technology for supporting practices of medical personnel.

### BACKGROUND

In recent years, as disclosed in Jpn. Pat. Appln. KOKAI Publication No. 2017-102885, a technique for managing the remaining amount of a medicine prescribed to a patient has been developed.

A doctor can grasp the usage of the patient's medicine by referring to the information on the remaining amount of a medicine prescribed to the patient. The doctor adjusts the type and amount of the next medicine to be prescribed to the patient according to the remaining amount of the medicine. Therefore, the information on the remaining amount of the medicine is important information for the doctor to diagnose the patient.

However, a doctor cannot appropriately diagnose a patient on whether or not the medicine is suitable for the patient, and whether or not the medicine is effective for the patient, etc. based only on the information of the remaining amount of the medicine. The doctor must collect information other than the information on the remaining amount of the medicine, and examine the patient. Thus, since doctors need time to grasp the patient's situation, efficiency has been desired in the examination practice.

### SUMMARY

A first aspect of the present invention is a data processing apparatus comprising a biological data acquisition unit that acquires biological data of a subject, a remaining amount estimation unit that estimates the remaining amount of a medicine prescribed to the subject, a display data generating unit that generates display data configuring a display screen including a biological data display indicating the biological data and a remaining medicine amount display indicating the remaining amount, and a display data output unit that outputs the display data.

According to the first aspect of the present invention, the medical personnel can, for example, easily grasp the blood pressure data and the remaining medicine amount of the subject as information necessary for examining a disease related to blood pressure at a glance. According to the combination of blood pressure data and remaining medicine amount of the subject, the medical personnel can easily grasp not only an urgency level for the subject to visit the medical personnel, but also whether or not the medicine is suitable for the subject, whether or not the medicine is working on the subject, and whether or not self-titration is working well on the subject. As a result, the medical personnel can efficiently examine the subject.

A second aspect of the present invention further comprises in the data processing apparatus of the first aspect a decision unit that determines an urgency level for the subject to visit the medical personnel based on the biological data and the remaining amount, in which the display data generating unit is configured to generate the display data that configures the display screen including urgency level display indicating the urgency level.

According to the second aspect of the present invention, the medical personnel can easily grasp the urgency level for the subject to visit the medical personnel. As a result, the medical personnel can efficiently examine the subject.

According to a third aspect of the present invention, in the data processing apparatus according to the first aspect, the display data generating unit is configured to generate the display data that configures the display screen including the remaining medicine amount display that varies depending on the remaining amount.

According to the third aspect of the present invention, the medical personnel can easily grasp the remaining medicine amount. As a result, the medical personnel can efficiently examine the subject.

According to a fourth aspect of the present invention, in the data processing apparatus according to the first aspect, the remaining amount estimation unit is configured to estimate the remaining amount based on the date on which the medicine is prescribed, the number of days for which the medicine is prescribed, and information regarding the medication input by the subject.

According to the fourth aspect of the present invention, the data processing apparatus can improve the estimation accuracy of the remaining medicine amount by using the information related to the medication input by the subject. As a result, the medical personnel can appropriately examine the subject by referring to the highly accurate information on the remaining medicine amount.

A fifth aspect of the present invention further comprises a decision unit that determines, from a plurality of candidates, an examination support message for supporting examination of the subject by the medical personnel based on the biological data and the remaining amount, in which the display data generating unit is configured to generate the display data configuring the display screen including the examination support message.

According to the fifth aspect of the present invention, the medical personnel can easily grasp matters to be considered according to the combination of the biological data and the remaining medicine amount at a glance with the support of the examination support message, and can thus efficiently examine the subject.

A sixth aspect of the present invention is a data processing method comprising a biological data acquisition process for acquiring biological data of a subject, a remaining amount estimation process for acquiring a remaining amount of a medicine prescribed to the subject, a display data generating process for generating display data configuring a display screen including a biological data display indicating the biological data and a remaining medicine amount display indicating the remaining amount, and a display data output process for outputting the display data.

According to the sixth aspect of the present invention, the data processing method can obtain the same effects as those of the first aspect described above.

A seventh aspect of the present invention is an information processing program that causes a computer to function as each unit included in a data processing apparatus according to any one of the first to fifth aspects.

According to the seventh aspect of the present invention, the data processing program can obtain the same effects as those of the first aspect described above.

According to the embodiments of the present invention, a technique to support practices of examining patients by medical personnel can be provided.

### BRIEF DESCRIPTION OF THE DRAWINGS

FIG. 1 is a block diagram showing an application example of a display screen server for medical personnel according to an embodiment.

FIG. 2 is a block diagram exemplifying a data transmission system including the display screen server for medical personnel according to the embodiment.

FIG. 3 is a block diagram exemplifying a hardware configuration of the display screen server for medical personnel according to the embodiment.

FIG. 4 is a block diagram exemplifying a software configuration of the display screen server for medical personnel according to the embodiment.

FIG. 5 is a diagram exemplifying an urgency level and an examination support message according to the embodiment.

FIG. 6 is a diagram showing a first display screen based on display data according to the embodiment.

FIG. 7 is a diagram showing a second display screen based on the display data according to the embodiment.

FIG. 8 is a diagram showing a third display screen based on the display data according to the embodiment.

FIG. 9 is a diagram showing a fourth display screen based on the display data according to the embodiment.

FIG. 10 is a diagram showing a fifth display screen based on the display data according to the embodiment.

FIG. 11 is a diagram showing a sixth display screen based on the display data according to the embodiment.

FIG. 12 is a diagram showing a seventh display screen based on the display data according to the embodiment.

FIG. 13 is a diagram showing an eighth display screen based on the display data according to the embodiment.

FIG. 14 is a diagram showing a ninth display screen based on the display data according to the embodiment.

FIG. 15 is a flowchart exemplifying an output operation of the display data by the display screen server for medical personnel according to the embodiment.

FIG. 16 is a flowchart exemplifying an urgency level decision operation by the display screen server for medical personnel according to the embodiment.

### DETAILED DESCRIPTION

Hereinafter, an embodiment according to an aspect of the present invention (hereinafter, also referred to as “present embodiment”) will be described with reference to the drawings. However, the present embodiment described below is merely an exemplification of the present invention in all respects. The data appearing in the present embodiment is described in a natural language. More specifically, it is specified by a pseudo language, a command, a parameter, a machine language, or the like that can be recognized by a computer. Hereinafter, elements that are the same as or similar to elements already described are denoted by the same or similar symbols, and redundant descriptions are basically omitted.

An object of the embodiments is to provide a data processing apparatus, a data processing method, and a data processing program that can support practices of examining patients by medical personnel.

§ 1 Application Example

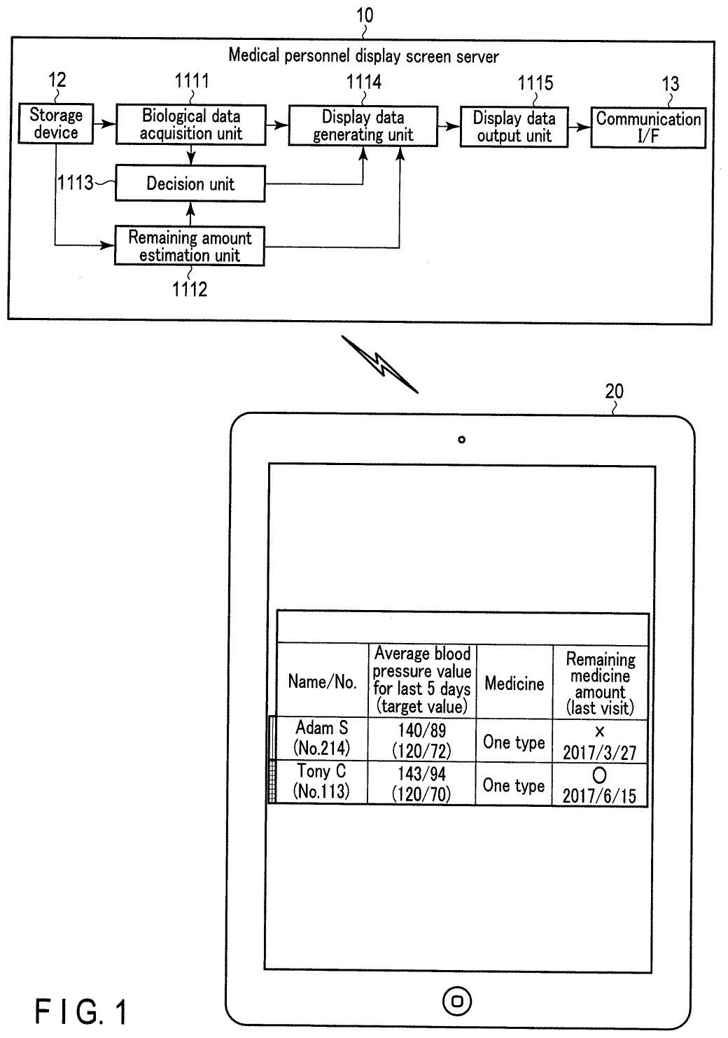

FIG. 1 is a diagram schematically showing an application example of a display screen server for medical personnel (hereinafter referred to as a medical personnel display screen server 10) according to the present embodiment.

The medical personnel display screen server 10 generates display data that configures a display screen for supporting practices of examining patients by medical personnel.

The medical personnel display screen server 10 includes a storage device 12, a communication interface 13, a biological data acquisition unit 1111, a remaining amount estimation unit 1112, a decision unit 1113, a display data generating unit 1114, and a display data output unit 1115.

The biological data acquisition unit 1111 acquires blood pressure data of a patient from the storage device 12. The biological data acquisition unit 1111 outputs the blood pressure data of the patient to the decision unit 1113 and the display data generating unit 1114.

The remaining amount estimation unit 1112 estimates the remaining medicine amount of the patient. The remaining amount estimation unit 1112 outputs information indicating the remaining medicine amount to the decision unit 1113 and the display data generating unit 1114.

Based on the blood pressure data and the remaining medicine amount, the decision unit 1113 determines the urgency level for the patient to visit the medical personnel. The decision unit 1113 outputs information indicating the urgency level to the display data generating unit 1114.

The display data generating unit 1114 generates display data that configures a display screen including a blood pressure data display indicating the blood pressure data and a remaining medicine amount display indicating the remaining medicine amount. The display data generating unit 1114 may generate display data that configures a display screen including an urgency level display indicating the urgency level in addition to the blood pressure data display and the remaining medicine amount display. The display data generating unit 1114 outputs the display data to the display data output unit 1115.

The display data output unit 1115 outputs the display data to the communication interface 13. The communication interface 13 transmits the display data to a portable terminal for medical personnel (hereinafter referred to as a medical personnel portable terminal 20) via a network.

The medical personnel portable terminal 20 configures a display screen based on the display data generated at the medical personnel display screen server 10 in the manner mentioned above. Medical personnel can examine the patient while checking the display screen.

As described above, according to the present embodiment, the medical personnel display screen server 10 can support practices of examining patients by the medical personnel.

§ 2 Configuration Example

<Data Transmission System>

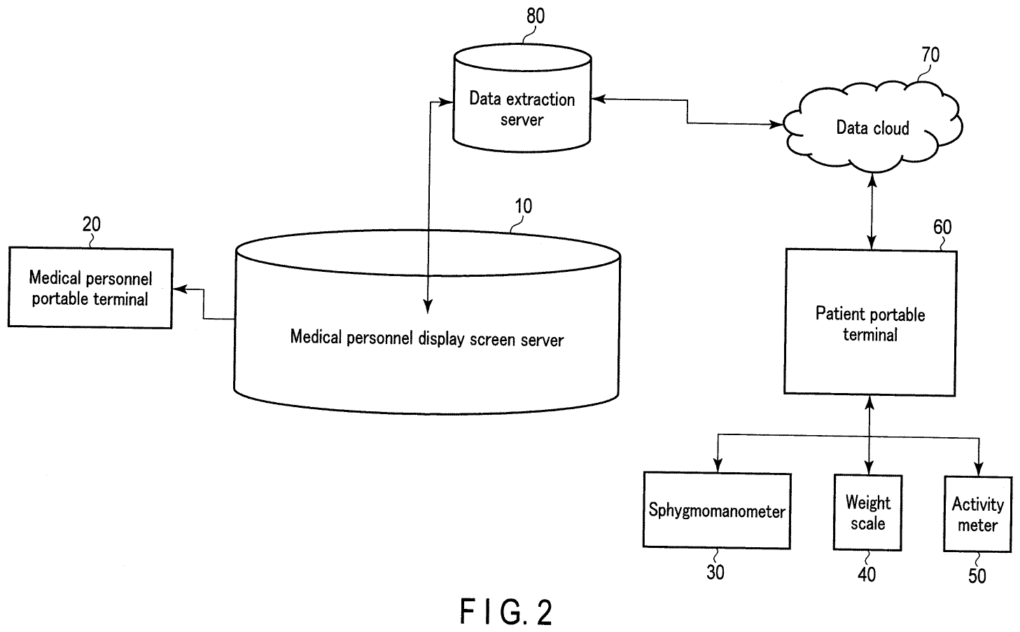

FIG. 2 is a block diagram exemplifying a data transmission system including the medical personnel display screen server 10 according to the present embodiment.

The data transmission system includes the medical personnel display screen server 10, the medical personnel portable terminal 20, a sphygmomanometer 30, a weight scale 40, an activity meter 50, a portable terminal for patients (hereinafter referred to as a patient portable terminal 60), a data cloud 70 and a data extraction server 80.

The medical personnel display screen server 10 generates display data that configures a display screen for supporting practices of examining patients by medical personnel, and outputs the display data to the medical personnel portable terminal 20. The medical personnel display screen server 10 is an example of a data processing apparatus. The configuration of the medical personnel display screen server 10 will be described later. For example, the medical person is a doctor; however, it is not limitative. A patient may be referred to as a subject.

The medical personnel portable terminal 20 is a smart phone or a tablet; however, it is not limited thereto. The medical personnel portable terminal 20 receives the display data from the medical personnel display screen server 10 via the network. For example, the network is a wireless local area network (WLAN) in a hospital or the Internet; however, it is not limited thereto. The medical personnel portable terminal 20 configures a display screen based on the display data.

The sphygmomanometer 30 is a sphygmomanometer capable of continuously measuring a patient's blood pressure per beat. For example, the sphygmomanometer 30 is a wearable sphygmomanometer. The sphygmomanometer 30 acquires blood pressure data by measuring the blood pressure of the patient.

The blood pressure data may include systolic blood pressure SBP and diastolic blood pressure DBP values and pulse rates; however, it is not limited thereto. Furthermore, the blood pressure data includes the measurement date and time of blood pressure. The measurement date and time is detected by a clock function mounted on the sphygmomanometer 30. The sphygmomanometer 30 may measure a patient's blood pressure from a pulse transit time (PTT), or by a tonometry method or other techniques. The sphygmomanometer 30 is an example of a biological information measuring device. Blood pressure data is an example of the biological data. The sphygmomanometer 30 transmits the blood pressure data of the patient to the patient portable terminal 60 using short-range wireless communication. For example, the short-range wireless communication is communication by Bluetooth (registered trademark); however, it is not limited thereto.

The weight scale 40 measures the weight of the patient. The weight scale 40 is an example of a biological information measuring device. Weight data is an example of the biological data. The weight scale 40 transmits the patient's weight data to the patient portable terminal 60 using short-range wireless communication.

The activity meter 50 continuously measures a patient's activity amount using acceleration signals representing accelerations in three directions orthogonal to each other. The activity meter 50 acquires activity data by measuring the activity amount of the patient. The activity data includes the measurement date and time of the activity amount. The measurement date and time is detected by a clock function mounted on the activity meter 50. The activity meter 50 transmits the activity data of the patient to the patient portable terminal 60 using short-range wireless communication.

The patient portable terminal 60 is a smart phone or a tablet; however, it is not limited thereto. The patient portable terminal 60 receives the blood pressure data from the sphygmomanometer 30 using short-range wireless communication. The patient portable terminal 60 transmits the blood pressure data to the data cloud 70 in association with the identification information of the patient who owns the patient portable terminal 60 via a network such as the Internet.

The patient portable terminal 60 receives the weight data from the weight scale 40 using short-range wireless communication. The patient portable terminal 60 generates BMI (Body Mass Index) data of the patient by referring to the weight data and pre-registered information on the height of the patient. The BMI data is an example of the biological data. The patient portable terminal 60 includes the date and time of receiving the weight data from the weight scale 40 in the BMI data as the date and time of when the patient measured his/her weight. The date and time of when the patient portable terminal 60 received the weight data from the weight scale 40 is detected by a clock function mounted on the patient portable terminal 60. The patient portable terminal 60 transmits the BMI data to the data cloud 70 in association with the identification information of the patient who owns the patient portable terminal 60 via a network such as the Internet.

The patient portable terminal 60 receives the activity data from the activity meter 50 using short-range wireless communication. The patient portable terminal 60 transmits the activity data to the data cloud 70 in association with the identification information of the patient who owns the patient portable terminal 60 via a network such as the Internet.

The data cloud 70 is a server that stores data related to a plurality of patients used to generate display data. The data cloud 70 stores data in association with the identification number of each patient. For example, the data cloud 70 stores blood pressure data, BMI data, and activity data.

The data extraction server 80 communicates with the medical personnel display screen server 10 and the data cloud 70 via a network such as the Internet. The data extraction server 80 extracts data used for generating the display data from the data cloud 70 in response to a request from the medical personnel display screen server 10. For example, the data extraction server 80 refers to the patient identification number included in the request from the medical personnel display screen server 10. The data extraction server 80 extracts data associated with this identification number from the data cloud 70. For example, the data extraction server 80 extracts blood pressure data, BMI data, and activity data from the data cloud 70. The data extraction server 80 transmits the data extracted from the data cloud 70 to the medical personnel display screen server 10. <Display Screen Server for Medical Personnel>

[Hardware Configuration]

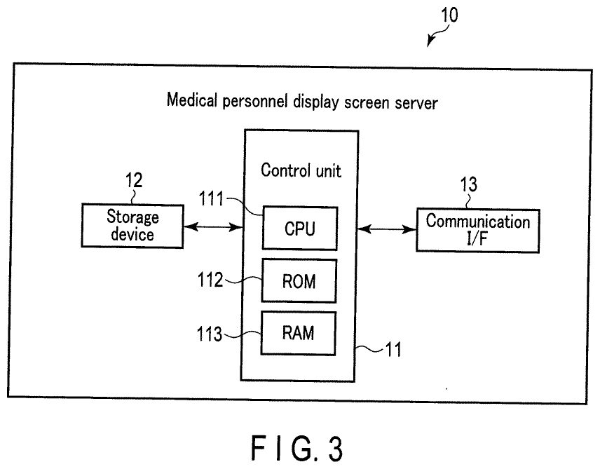

FIG. 3 is a diagram schematically showing an example of a hardware configuration of the medical personnel display screen server 10.

The medical personnel display screen server 10 is a computer in which a control unit 11, a storage device 12, and a communication interface 13 are electrically connected. In FIG. 3, the communication interface is described as “communication I/F”.

The control unit 11 controls the operation of each unit of the medical personnel display screen server 10. The control unit 11 includes a CPU (Central Processing Unit) 111, a ROM (Read Only Memory) 112, a RAM (Random Access Memory) 113, and the like. The CPU 111 is an example of a processor. The CPU 111 expands in the RAM 113 a program stored in the storage device 12 for causing the medical personnel display screen server 10 to function. The control unit 11 is able to execute various types of information processing by the CPU 111 interpreting and executing the program expanded in the RAM 113. For example, the control unit 11 is able to execute the biological data acquisition unit 1111, the remaining amount estimation unit 1112, the decision unit 1113, the display data generating unit 1114, and the display data output unit 1115 described under the item of the software configuration by the CPU 111 interpreting and executing the program expanded in the RAM 113.

The storage device 12 is a so-called auxiliary storage device. For example, the storage device 12 is an HDD (Hard Disk Drive); however, it is not limited thereto. The storage device 12 stores a program executed by the control unit 11 and various data used by the control unit 11. The program can also be referred to as a command for operating the control unit 11.

The storage device 12 stores various information regarding a patient. For example, the storage device 12 stores a blood pressure target value set for the patient in association with the patient identification number. The blood pressure target value is based on information transmitted from the medical personnel portable terminal 20. An example will be described. The medical personnel refer to the blood pressure data of the patient and set the blood pressure target value of the patient on the medical personnel mobile terminal 20. The medical personnel portable terminal 20 transmits setting information indicating the blood pressure target value and the patient identification number to the medical personnel display screen server 10. The medical personnel display screen server 10 stores the blood pressure target value set for the patient in the storage device 12 in association with the patient identification number based on the setting information.

The storage device 12 includes a patient database and a remaining medicine amount database.

The patient database accumulates blood pressure data, BMI data, and activity data of each patient acquired from the data cloud 70 via the data extraction server 80 in association with the identification number of each patient.

The remaining medicine amount database accumulates data relating to the remaining amount of a medicine prescribed to each patient (hereinafter also referred to as remaining medicine amount) in association with the identification number of each patient. The data regarding the remaining medicine amount includes the type of medicine, the date on which the medicine is prescribed, the number of days for which the medicine is prescribed, and information on the medication input by the patient. The date on which the medicine is prescribed corresponds to the date of the patient's last visit to the hospital.

The type of medicine, the date on which the medicine is prescribed, and the number of days for which the medicine is prescribed are based on information transmitted from the medical personnel portable terminal 20. An example will be described. The medical personnel set the type of medicine to be prescribed to the patient and the number of days for which the medicine is prescribed on the medical personnel portable terminal 20. The medical personnel portable terminal 20 acquires the patient identification number and the date on which the medicine is prescribed, in addition to the type of medicine and the number of days for which the medicine is prescribed. The medical personnel portable terminal 20 transmits to the medical personnel display screen server 10 setting information indicating the type of medicine, the number of days for which the medicine is prescribed, the patient identification number, and the date on which the medicine is prescribed. The medical personnel display screen server 10 updates the remaining medicine amount database based on the setting information.

The information related to the medication is information indicating whether or not the patient is taking the medicine in accordance with the specified dosage and administration. The information related to the medication is based on information transmitted from the patient portable terminal 60. An example will be described. For every predetermined period, the patient inputs in the patient portable terminal 60 that the medicine was taken in accordance with the specified dosage and administration within this period. For example, every day, the patient inputs that the medicine was taken for the day in the patient portable terminal 60. Instead of this, for every predetermined period, the patient may also input in the patient portable terminal 60 that the medicine was forgotten to be taken in accordance with the specified dosage and administration within the predetermined period. For example, every day, the patient inputs that the medicine was forgotten to be taken for the day in the patient portable terminal 60. The patient portable terminal 60 transmits information input by the patient to the medical personnel display screen server 10. The medical personnel display screen server 10 updates the remaining medicine amount database based on the input information.

The communication interface 13 includes various wireless communication modules for mobile communication (3G, 4G, etc.) and WLAN, etc. The communication interface 13 communicates with the medical personnel portable terminal 20, the patient portable terminal 60, and the data extraction server 80.

In addition, regarding the specific hardware configuration of the medical personnel display screen server 10, components can be omitted, replaced, and added as appropriate according to the embodiment. For example, the control unit 11 may include a plurality of processors.

<Display Screen Server for Medical Personnel>

[Software Configuration]

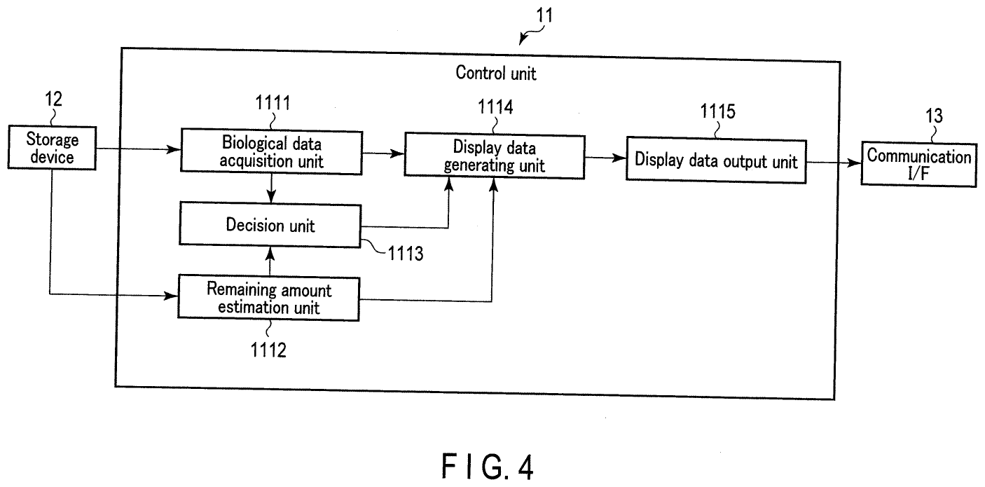

FIG. 4 is a diagram schematically showing an example of a software configuration of the medical personnel display screen server 10.

The control unit 11 implements the biological data acquisition unit 1111, the remaining amount estimation unit 1112, the decision unit 1113, the display data generating unit 1114, and the display data output unit 1115. For simplification of description, here, an example in which the control unit 11 generates display data that configures a display image including various displays of one patient will be described. The control unit 11 can also generate display data that configures a display image including various displays of each of a plurality of patients in the same manner.

The biological data acquisition unit 1111 will be described.

The biological data acquisition unit 1111 acquires blood pressure data of a patient in the manner exemplified below. The biological data acquisition unit 1111 acquires blood pressure data associated with the patient identification number from the patient database included in the storage device 12. The biological data acquisition unit 1111 outputs the blood pressure data of the patient to the decision unit 1113 and the display data generating unit 1114.

The remaining amount estimation unit 1112 will be described.

The remaining amount estimation unit 1112 estimates the remaining amount of a medicine prescribed to the patient in the manner exemplified below. For example, the remaining amount estimation unit 1112 estimates the amount of remaining medicine based on the date on which the medicine is prescribed, the number of days for which the medicine is prescribed, and the information related to the medication input by the patient. In this example, first, the remaining amount estimation unit 1112 calculates the number of elapsed days from the date when the medicine was prescribed to the present. Next, the remaining amount estimation unit 1112 calculates the number of days that the patient has forgotten to take the medicine between the date when the medicine was prescribed and the present based on the information related to the medication. The remaining amount estimation unit 1112 subtracts the number of elapsed days and the number of days the patient has forgotten to take the medicine from the number of days for which the medicine is prescribed, and estimates the amount of medicine for such remaining days as the remaining medicine amount.

The remaining amount estimation unit 1112 outputs information indicating the remaining medicine amount to the decision unit 1113 and the display data generating unit 1114.

The decision unit 1113 will be described.

As exemplified below, the decision unit 1113 decides the urgency level regarding the patient's visit to the medical personnel based on the blood pressure data and the remaining medicine amount. In this example, the decision unit 1113 refers to the blood pressure data of the patient from the biological data acquisition unit 1111 and the information indicating the remaining medicine amount from the remaining amount estimation unit 1112. An example of determining the urgency level by the decision unit 1113 will be described later. The decision unit 1113 outputs information indicating the urgency level to the display data generating unit 1114.

Furthermore, in the manner exemplified below, based on the blood pressure data and the remaining medicine amount, the decision unit 1113 selects (determines) from among a plurality of candidates an examination support message for supporting the patient's examination by the medical personnel. In this example, the decision unit 1113 refers to the blood pressure data of the patient from the biological data acquisition unit 1111, the information indicating the remaining medicine amount from the remaining amount estimation unit 1112, and the plurality of examination support messages stored in the storage device 12. The storage device 12 stores a combination of the blood pressure value and the remaining medicine amount in association with each of a plurality of preset examination support messages. The blood pressure value described in the present embodiment refers to both the systolic blood pressure SBP value and the diastolic blood pressure DBP value. An example of selecting the examination support message by the decision unit 1113 will be described later. The decision unit 1113 outputs information indicating the examination support message to the display data generating unit 1114.

Here, the decision unit 1113 determines the urgency level and selects the examination support message; however, this may also be performed by another element mounted on the control unit 11.

The display data generating unit 1114 will be described.

The display data generating unit 1114 generates various display data in the manner exemplified below. For example, the display data generating unit 1114 generates display data that configures a display screen including a blood pressure data display indicating the blood pressure data and a remaining medicine amount display indicating the remaining medicine amount. The blood pressure data display and the remaining medicine amount display are associated with a patient display. The patient display is, for example, a patient name and a patient number. The blood pressure data display may indicate an average blood pressure value for the last five days.

The display data generating unit 1114 may generate display data that configures a display image including the remaining medicine amount display that varies depending on the remaining medicine amount. For example, the remaining medicine amount display is displayed in three stages corresponding to the remaining medicine amount; however, it may also be displayed in two stages or four or more stages. For example, in the case where there is no remaining medicine amount, the remaining medicine amount display shows “×”. In the case where the remaining medicine amount is greater than zero days and less than ten days, the remaining medicine amount display shows “Δ”. In the case where the remaining medicine amount is ten days or more, the remaining medicine amount display shows “◯”. The display mode is not limited. Therefore, instead of the above-described symbols, the remaining medicine amount display may show colors that vary according to the remaining medicine amount, or may show patterns according to the remaining medicine amount.

The display data generating unit 1114 may generate display data that configures a display screen including an urgency level display indicating the urgency level in addition to the blood pressure data display and the remaining medicine amount display. In this example, the blood pressure data display, the remaining medicine amount display, and the urgency level display are associated with the patient display. The urgency level display shows colors that vary according to the urgency level. The display mode is not limited. Therefore, instead of colors, the urgency level display may show symbols that vary according to the urgency level.

The display data generating unit 1114 may generate display data that configures a display image including at least one display among a target value display, a medicine number display, a last visit display, an examination support message display, and a remaining medicine amount calculation formula display, in addition to the blood pressure data display, the remaining medicine amount display, and the urgency level display. These displays are associated with the patient display. The target value display is based on the blood pressure target value information set for the patient stored in the storage device 12. The medicine number display is based on the type of medicine prescribed to the patient accumulated in the remaining medicine amount database. The last visit display is based on the date on which the medicine was prescribed accumulated in the remaining medicine amount database. The examination support message display is based on the examination support message selected by the decision unit 1113. The remaining medicine amount calculation formula display is based on the number of days for which the medicine is prescribed, the number of elapsed days, and the number of days the patient has forgotten to take the medicine, which are used for estimating the remaining medicine amount.

Furthermore, the display data generating unit 1114 may generate display data that configures a display screen showing details for each patient. The display screen showing details includes a blood pressure data display, a BMI data display showing BMI data, an activity data display showing activity data, and a display of setting fields for medical personnel, etc. The blood pressure data display includes a display of the average blood pressure value for the last five days and a display of the average blood pressure value for the five days from the last visit. Furthermore, the blood pressure data display includes a display of a graph showing changes in blood pressure for each month, week or day. The BMI data display includes a display of a graph showing changes in BMI for each month, week or day. The activity data display includes a display of a graph showing changes in activity amount for each month, week or day. The display of setting fields for medical personnel is configured so that information related to the patient's examination can be set. For example, the display of setting fields for medical personnel is configured so that the blood pressure value at a workplace, the target value of the patient's blood pressure, the blood pressure value serving as a threshold for issuing a warning to the medical personnel and the patient, the type of medicine to be prescribed, and the number of days for which the medicine is prescribed can be set.

The display data generating unit 1114 may also generate display data that configures a display screen other than these.

The display screen based on the display data will be described later.

The display data generating unit 1114 outputs the display data to the display data output unit 1115.

The display data output unit 1115 will be described.

The display data output unit 1115 outputs the display data in the manner exemplified below. The display data output unit 1115 receives the display data from the display data generating unit 1114. The display data output unit 1115 outputs the display data to the communication interface 13. The communication interface 13 transmits the display data to the medical personnel portable terminal 20 via a network.

<Urgency Level and Examination Support Message>

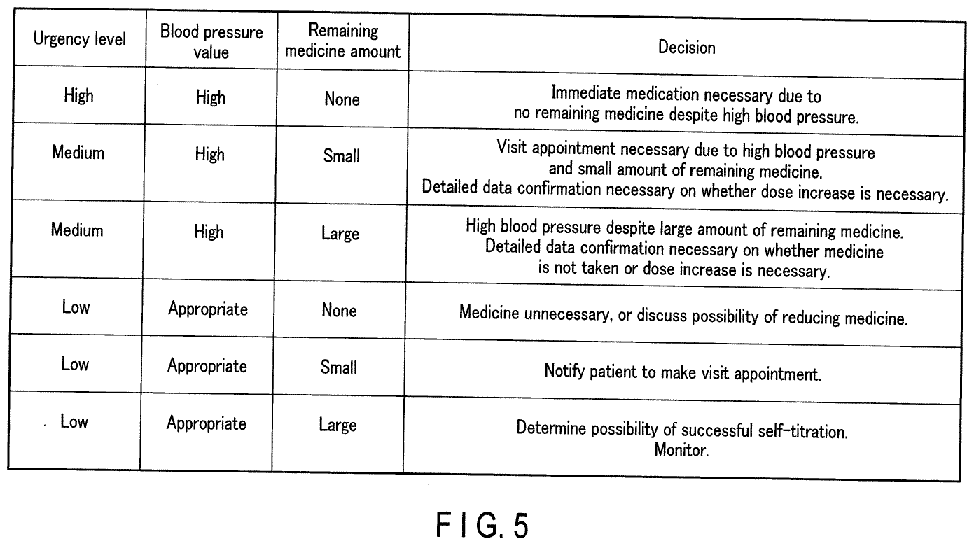

FIG. 5 is a diagram exemplifying the urgency level and the examination support message.

First, an example of determining the urgency level by the decision unit 1113 will be described.

Here, an example in which the decision unit 1113 determines the urgency level in three stages (high, medium, and low) will be described. The decision unit 1113 may also determine the urgency level in two stages or in four or more stages.

First, the decision unit 1113 acquires the blood pressure data from the biological data acquisition unit 1111. The decision unit 1113 acquires a blood pressure value from the blood pressure data. For example, the decision unit 1113 acquires the blood pressure value by calculating the average value of the blood pressure values for the last five days. The blood pressure value used by the decision unit 1113 for deciding the urgency level is not limited, therefore, may be an average value for the last 10 days, or an average value for the previous day, etc. The decision unit 1113 determines whether or not the blood pressure value satisfies a reference. The reference is arbitrarily set to evaluate whether or not the blood pressure value is appropriate. For example, the reference is set so that the value of systolic blood pressure SBP is lower than 140 mmHg, and the value of diastolic blood pressure DBP is lower than 90 mmHg; however, the values are not limited thereto. In the case where the blood pressure value satisfies the reference, the decision unit 1113 determines that the patient's blood pressure value is appropriate. On the other hand, in the case where the blood pressure value does not satisfy the reference, the decision unit 1113 determines that the patient's blood pressure value is high.

Next, the decision unit 1113 determines the remaining medicine amount. Here, the decision unit 1113 refers to a reference value that is arbitrarily set in order to evaluate the remaining medicine amount. For example, the reference value is the amount of 10 days' of medicine; however, the amount is not limited thereto. In the case where the remaining medicine amount is zero, the decision unit 1113 determines that there is no remaining medicine amount. In the case where the remaining medicine amount is greater than zero and lower than the reference value, the decision unit 1113 determines that the remaining medicine amount is small. In the case where the remaining medicine amount is greater than or equal to the reference value, the decision unit 1113 determines that the remaining medicine amount is large.

The decision unit 1113 determines the urgency level based on the combination of the blood pressure value and the remaining medicine amount classified in the manner described above. In the case where the blood pressure value is high, and there is no remaining medicine amount, the decision unit 1113 determines that the urgency level is “high”. In the case where the blood pressure value is high, and there is a remaining medicine amount, the decision unit 1113 determines that the urgency level is medium. In the case where the blood pressure value is appropriate regardless of the remaining medicine amount, the decision unit 1113 determines that the urgency level is “low”.

Instead of determining the urgency level based on the blood pressure data and the remaining medicine amount, the decision unit 1113 may determine the urgency level based on the blood pressure data. Here, an example in which the decision unit 1113 determines the urgency level in three stages (high, medium, and low) will be described. The decision unit 1113 may also determine the urgency level in two stages or in four or more stages.

The decision unit 1113 acquires the blood pressure value from the blood pressure data in the manner described above. In the case where the value of the systolic blood pressure SBP is 160 mmHg or higher, or the value of the diastolic blood pressure DBP is 100 mmHg or higher, the decision unit 1113 determines that the urgency level is “high”. In the case where the value of the systolic blood pressure SBP is 140 mmHg or higher and is lower than 160 mmHg, or in the case where the value of the diastolic blood pressure DBP is 90 mmHg or higher and is lower than 100 mmHg, the decision unit 1113 determines that the urgency level is “medium”. In the case where the value of the systolic blood pressure SBP is lower than 140 mmHg, and the value of the diastolic blood pressure DBP is lower than 90 mmHg, the decision unit 1113 determines that the urgency level is “low”.

Next, an example of selecting an examination support message by the decision unit 1113 will be described.

Based on the combination of the blood pressure value and the remaining medicine amount classified in the manner described above, the decision unit 1113 selects an examination support message as shown in the item “decision” in FIG. 5 from a plurality of candidates. In the case where the blood pressure value is high, and there is no remaining medicine amount, the decision unit 1113 selects an examination support message that prompts the medical personnel to consider that urgent medication is necessary. In the case where the blood pressure value is high, and the remaining medicine amount is small, the decision unit 1113 selects an examination support message that prompts the medical personnel to consider whether or not the patient needs to make a visit appointment, and whether or not the dose needs to be increased. In the case where the blood pressure value is high, and the remaining medicine amount is large, the decision unit 1113 selects an examination support message that prompts the medical personnel to consider whether or not the patient is taking the medicine, or whether or not the dose needs to be increased. In the case where the blood pressure value is appropriate, and there is no remaining medicine amount, the decision unit 1113 selects an examination support message that prompts the medical personnel to consider that the medicine is unnecessary, or consider the possibility of reducing the dose. In the case where the blood pressure value is appropriate, and the remaining medicine amount is small, the decision unit 1113 selects an examination support message that prompts the medical personnel to consider notifying the patient to make a visit appointment. In the case where the blood pressure value is appropriate, and the remaining medicine amount is large, the decision unit 1113 selects an examination support message that prompts the medical personnel to consider the possibility of the patient's self-titration working well, and consider monitoring such. Here, self-titration means to have the patient refer to his/her own blood pressure data and adjust the dose and interval of taking the medicine according to his/her own judgment.

As described above, the decision unit 1113 selects a different examination support message depending on the remaining medicine amount even when the urgency level is “medium” or “low”. This is because the matters desired to be considered by the medical personnel differ depending on the combination of the blood pressure value and the remaining medicine amount.

<Display Screen>

The medical personnel portable terminal 20 configures various display screens based on various display data generated by the medical personnel display screen server 10. Here, some examples of the display screen based on the display data will be described.

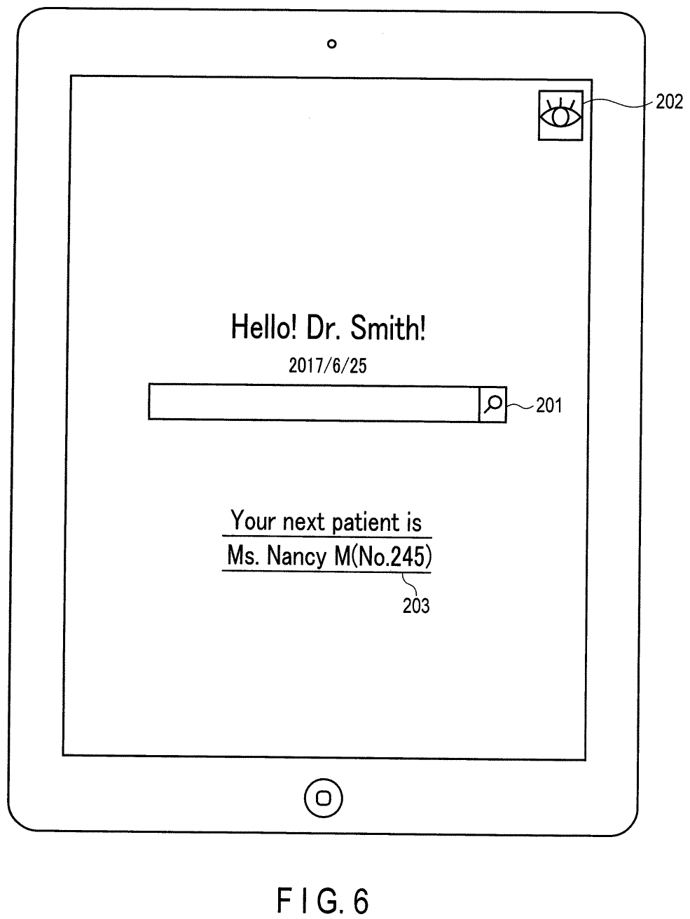

FIG. 6 to FIG. 14 are diagrams schematically showing examples of display screens based on the display data.

FIG. 6 shows a first display screen based on the display data. The first display screen includes a search box display 201, a link display 202, and a link display 203. The search box display 201 is configured in a manner that a keyword such as a patient name, a patient number, or an urgency level can be input in order to search for a specific patient among all patients. The link display 202 is configured to link to a display screen showing a list of all patients in response to being selected by the medical personnel. The link display 203 is configured to link to a display screen showing details of a patient to be examined next in response to being selected by the medical personnel.

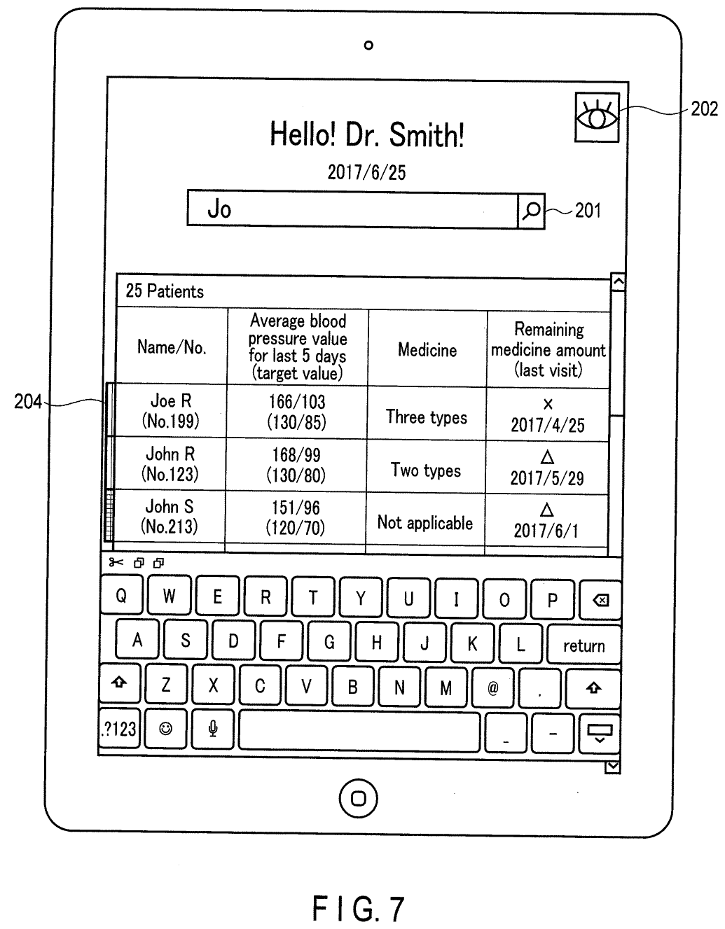

FIG. 7 shows a second display screen based on the display data. The second display screen transitions from the first display screen in response to a keyword being input to the search box display 201 of the first display screen. The second display screen shows a list of patients including the keyword input in the search box display 201 in part of the name. The second display screen includes the blood pressure data display, the remaining medicine amount display, an urgency level display 204, the target value display, the medicine number display, and the last visit display. These displays are associated with the patient display. For example, the remaining medicine amount display shows symbols that vary according to the remaining medicine amount. For example, the urgency level display 204 shows colors that vary according to the urgency level. The second display screen should at least include the blood pressure data display and the remaining medicine amount display, and may not have to include all of the above-described displays. In the same manner as the second display screen, third to fifth display screens to be described later should at least include the blood pressure data display and the remaining medicine amount display, and may not have to include all of the above-described displays.

The second display screen may include a display of an examination support message that is displayed in response to a cursor operation by the medical personnel. For example, in response to the medical personnel placing the cursor on a field of any patient, the display of the examination support message shows the examination support message selected for this patient. By the support of the examination support message, the medical personnel can efficiently examine the patient. In the same manner as the second display screen, the third to fifth display screens described later may also include a display of the examination support message.

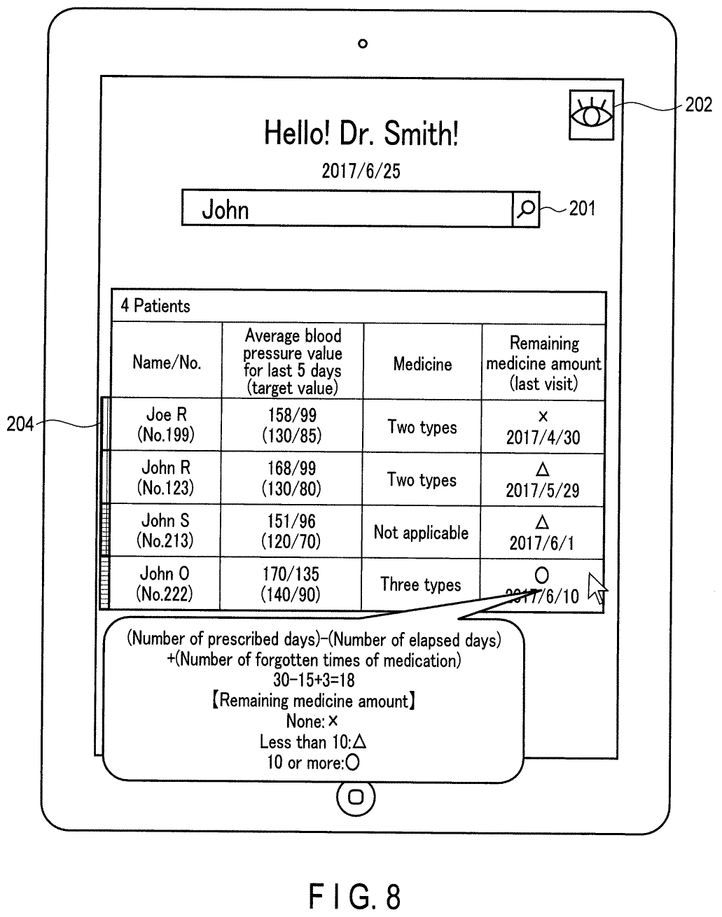

FIG. 8 shows the third display screen based on the display data. In the same manner as the second display screen, the third display screen shows a list of patients including a keyword input in the search box display 201 in a part of the name. In the same manner as the second display screen, the third display screen includes the blood pressure data display, the remaining medicine amount display, the urgency level display 204, the target value display, the medicine number display, and the last visit display. Furthermore, the third display screen includes a display of a remaining medicine amount calculation formula displayed in response to a cursor operation by the medical personnel. For example, in response to the medical personnel placing the cursor on the field of any patient, the display of the remaining medicine amount calculation formula shows the estimated remaining medicine amount for this patient. The display of the remaining medicine amount calculation formula indicates the number of days for which the medicine is prescribed, the number of elapsed days, and the number of days the patient has forgotten to take the medicine, which are used for estimating the remaining medicine amount, in addition to the remaining medicine amount.

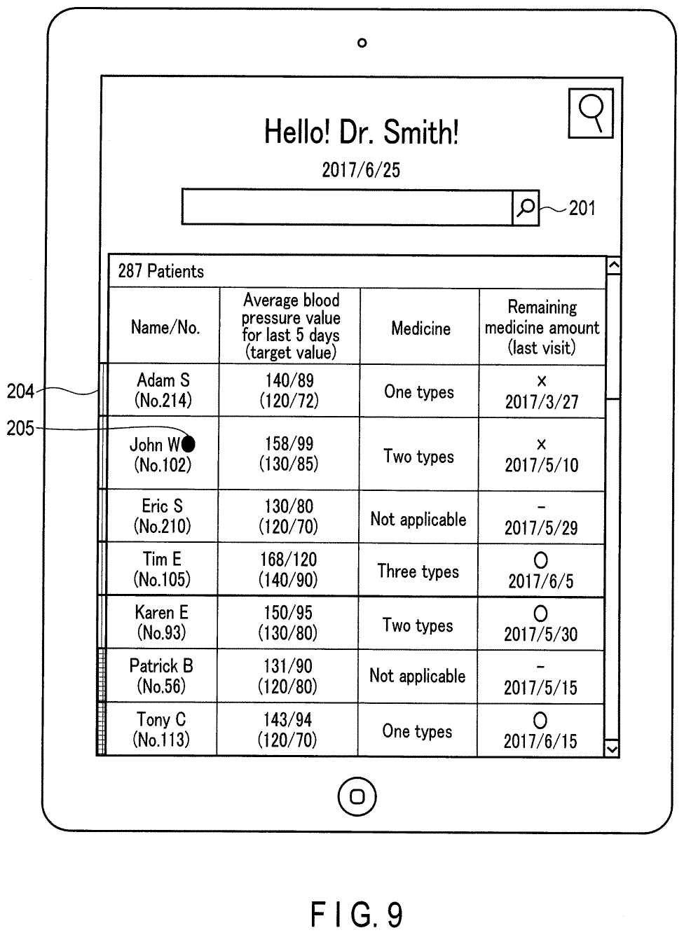

FIG. 9 shows the fourth display screen based on the display data. The fourth display screen transitions from the first display screen in response to the link display 202 on the first display screen being selected. The fourth display screen shows a list of all patients. The fourth display screen may transition from the second display screen or the third display screen in response to the link display 202 included in the second display screen or the third display screen being selected. In the same manner as the second display screen, the fourth display screen includes the blood pressure data display, the remaining medicine amount display, the urgency level display 204, the target value display, the medicine number display, and the last visit display. Furthermore, the fourth display screen includes a warning display 205. The warning display 205 indicates that the blood pressure value exceeds a threshold set by the medical personnel. The medical personnel can confirm the information of all patients by scrolling the fourth display screen.

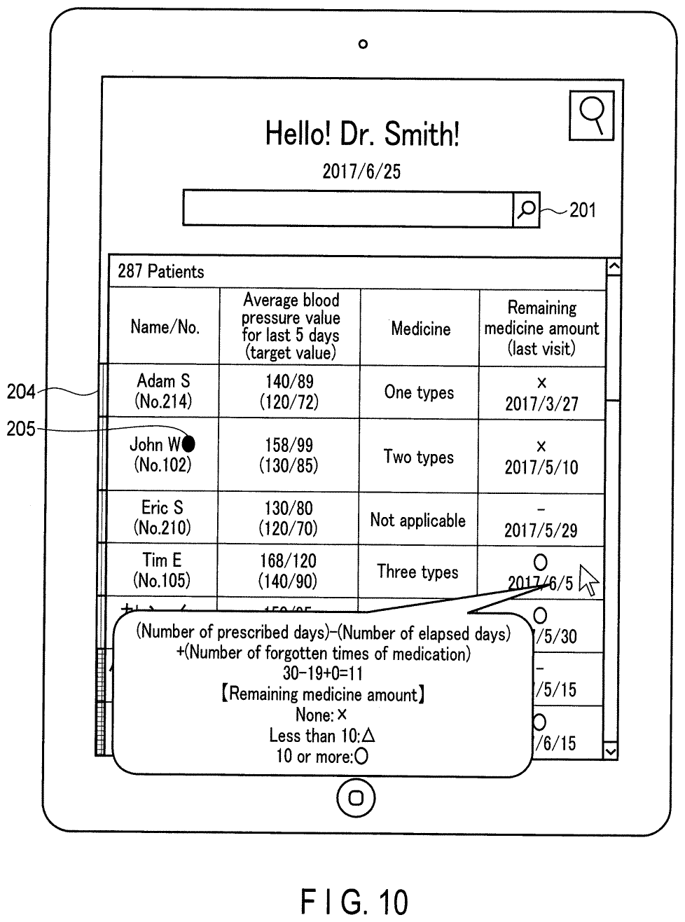

FIG. 10 shows the fifth display screen based on the display data. In the same manner as the fourth display screen, the fifth display screen shows a list of all patients. Furthermore, in the same manner as the third display screen, the fifth display screen includes a display of the remaining medicine amount calculation formula displayed in response to the cursor operation by the medical personnel.

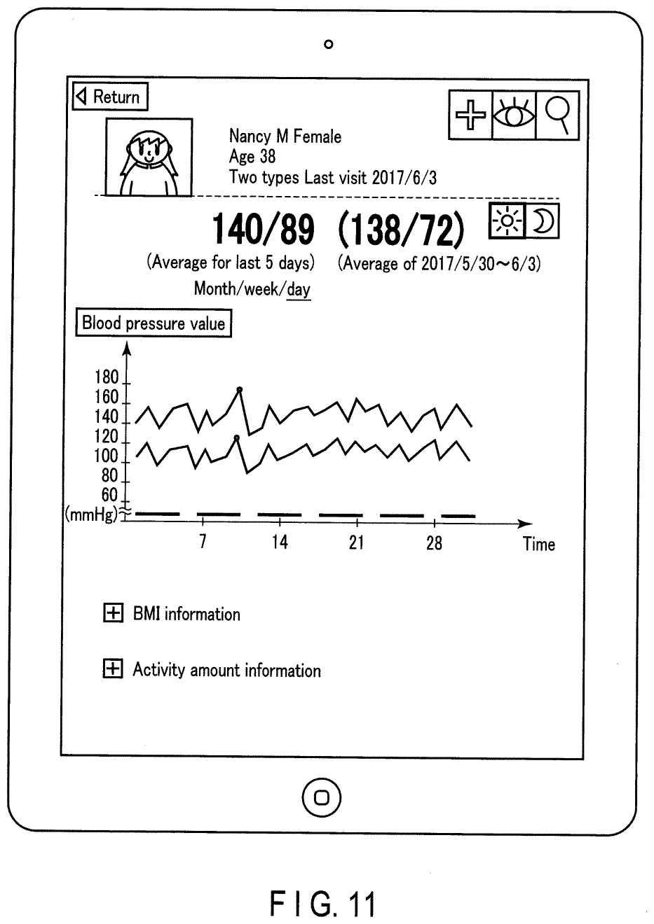

FIG. 11 shows a sixth display screen based on the display data. The sixth display screen shows details of the patient to be diagnosed. The sixth display screen transitions from the first display screen in response to the medical personnel selecting the link display 203 on the first display screen. The sixth display screen includes the blood pressure data display including the average blood pressure value for the last five days, the average blood pressure value for five days from the last visit, and a graph showing the change in blood pressure by month, week or day.

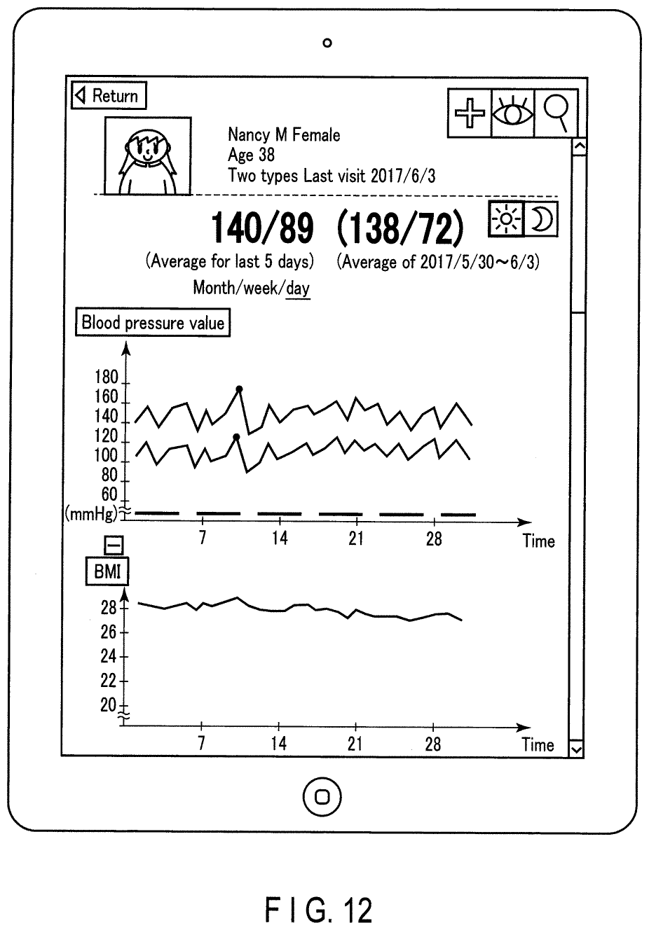

FIG. 12 shows a seventh display screen based on the display data. The seventh display screen shows details of the patient to be diagnosed. The seventh display screen includes the BMI data display that is displayed in response to the medical personnel selecting a BMI information field on the sixth display screen. The BMI data display includes a graph showing the change in BMI by month, week or day.

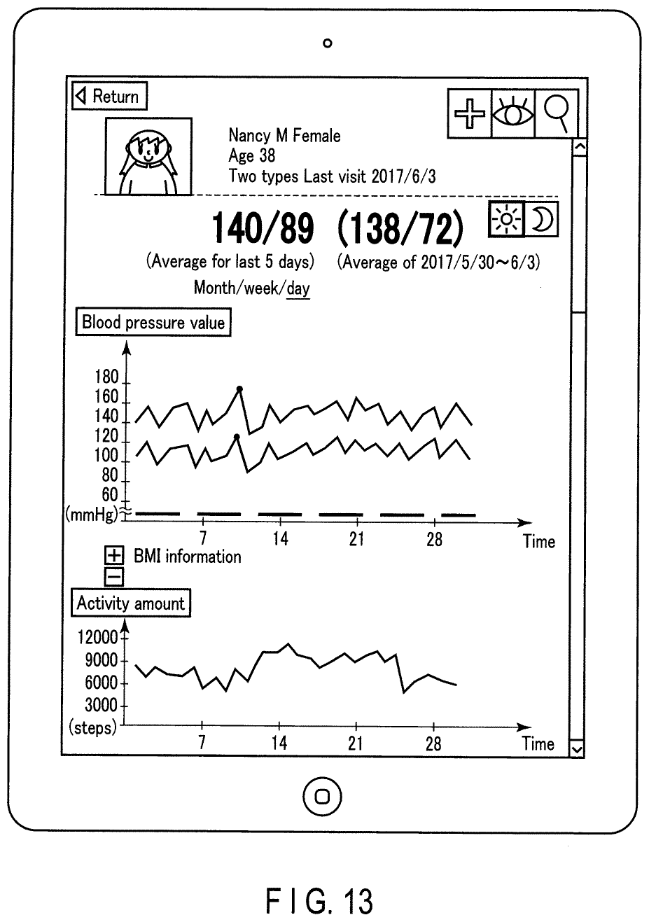

FIG. 13 shows an eighth display screen based on the display data. The eighth display screen shows details of the patient to be diagnosed. The eighth display screen includes the activity data display that is displayed in response to the medical personnel selecting an activity amount information field on the sixth display screen. The activity data display includes a graph showing the change in activity amount by month, week or day.

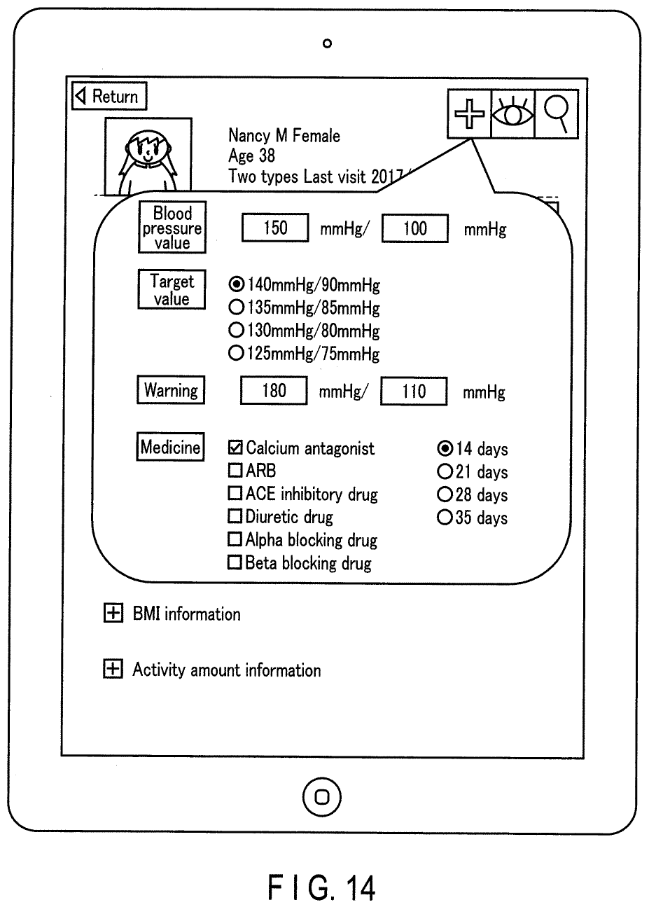

FIG. 14 shows a ninth display screen based on the display data. The ninth display screen shows details of the patient to be diagnosed. The ninth display screen includes a display of a medical personnel setting field displayed in response to a predetermined field on the sixth display screen being selected. The medical personnel can set various information according to the diagnosis of the patient. For example, the medical personnel can set a blood pressure value at a workplace, a target value of a patient's blood pressure, a blood pressure value serving as a threshold for issuing a warning to the medical personnel and the patient, the type of medicine to be prescribed, and the number of days for which the medicine is prescribed. The medical personnel portable terminal 20 transmits the information set in the medical personnel setting field to the medical personnel display screen server 10.

§ 3 Example of Operation

<Display Screen Server for Medical Personnel>

(Display Data Output Operation)

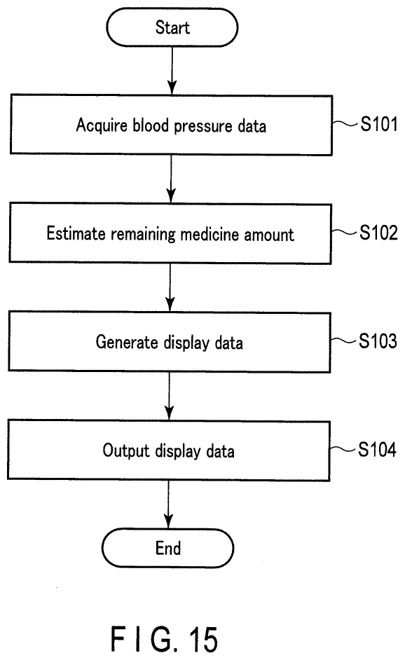

FIG. 15 is a flowchart showing an example of an output operation of the display data by the medical personnel display screen server 10. The processing procedure described below is merely an example, and each processing may be changed to the extent necessary. Furthermore, in the processing procedure described below, steps can be omitted, replaced, and added as appropriate according to the embodiment.

As exemplified, the biological data acquisition unit 1111 acquires blood pressure data of the patient (step S101).

As exemplified, the remaining amount estimation unit 1112 estimates the remaining medicine amount (step S102).

As exemplified, the display data generating unit 1114 generates display data configuring a display image including the blood pressure data display indicating the blood pressure data and the remaining medicine amount display indicating the remaining medicine amount (step S103). The display data generating unit 1114 may generate the display data configuring a display screen including another display in addition to or instead of the blood pressure data display and the remaining medicine amount display.

As exemplified, the display data output unit 1115 outputs the display data (step S104).

<Display Screen Server for Medical Personnel>

(Urgency Level Decision Operation)

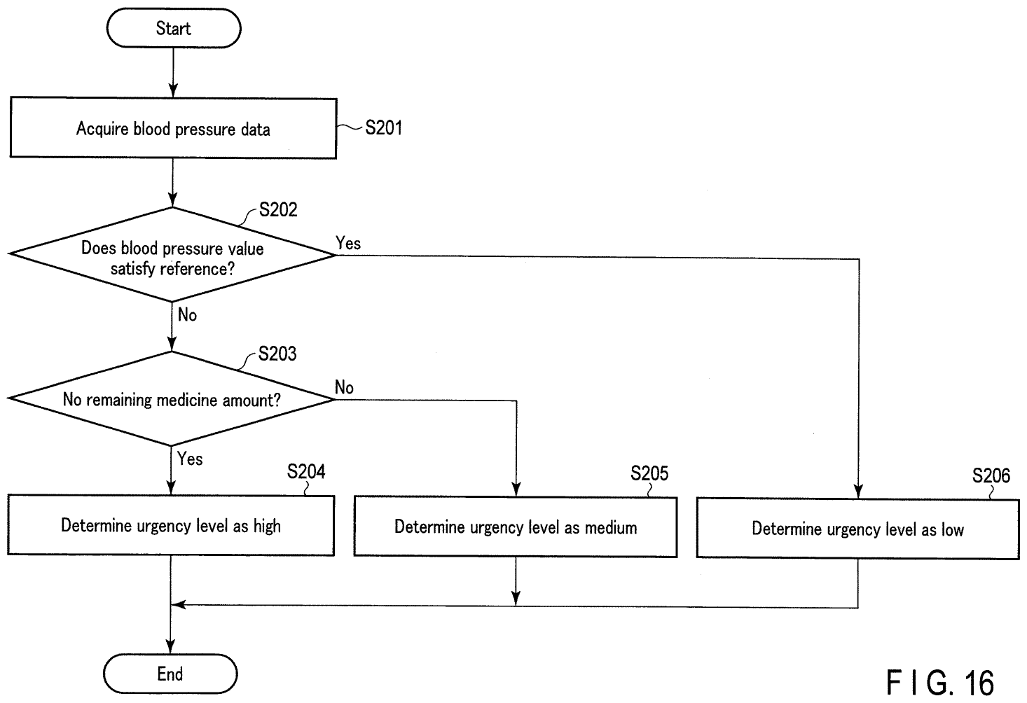

FIG. 16 is a flowchart showing an example of an urgency level decision operation performed by the medical personnel display screen server 10. The processing procedure described below is merely an example, and each processing may be changed to the extent necessary. Furthermore, in the processing procedure described below, steps can be omitted, replaced, and added as appropriate according to the embodiment.

As exemplified, the decision unit 1113 acquires the blood pressure data of the patient (Step S201).

As exemplified, the decision unit 1113 determines whether or not the blood pressure value satisfies the reference (step S202).

In the case where the blood pressure value does not satisfy the reference (step S202, No), the decision unit 1113 determines whether or not there is a remaining medicine amount as exemplified (step S203). In the case where there is no remaining medicine amount (step S203, Yes), the decision unit 1113 determines that the urgency level is “high” (step S204). In the case where there is remaining medicine amount (step S203, No), the decision unit 1113 decides that the urgency level is “medium” (step S205).

In the case where the blood pressure value satisfies the reference (step S202, Yes), the decision unit 1113 decides that the urgency level is “low” (step S206).

[Action/Effect]

As described above, in the present embodiment, the medical personnel display screen server 10 generates the display data that configures the display screen including the blood pressure data display and the remaining medicine amount display.

Thereby, the medical personnel can easily grasp at a glance the blood pressure data and the remaining medicine amount of the patient, which is information necessary for examining a disease related to blood pressure. According to the combination of the patient's blood pressure data and the remaining medicine amount, the medical personnel can easily grasp not only the urgency level regarding the patient's visit to the medical personnel, but also whether or not the medicine is right for the patient, whether or not the medicine is working for the patient, and whether or not self-titration is working for the patient, etc. As a result, the medical personnel can efficiently examine the patient.

Furthermore, in this embodiment, the medical personnel display screen server 10 generates display data that configures the display screen including the urgency level display.

Thereby, the medical personnel can easily grasp the urgency level regarding a patient's visit to the medical personnel. As a result, the medical personnel can efficiently examine the patient.

Furthermore, in the present embodiment, the medical professional display screen server 10 generates display data that configures the display screen including the remaining medicine amount display that varies according to the remaining medicine amount.

Thereby, the medical personnel can easily grasp the remaining medicine amount. As a result, the medical personnel can efficiently examine the patient.

Furthermore, in the present embodiment, the medical personnel display screen server 10 estimates the remaining medicine amount based on the date on which the medicine is prescribed, the number of days for which the medicine is prescribed, and the information related to the medication input by the patient.

Thereby, the medical personnel display screen server 10 can improve the estimation accuracy of the remaining medicine amount by using the information related to the medication input by the patient. As a result, the medical personnel can appropriately examine the patient by referring to the highly accurate information on the remaining medicine amount.

Furthermore, in the present embodiment, the medical personnel display screen server 10 generates display data that configures the display screen including the examination support message for supporting examination of patients by the medical personnel.

Therefore, since matters to be considered according to the combination of the blood pressure data and the remaining medicine amount can be easily grasped at a glance with the support of the examination support message, the medical personnel can examine the patient efficiently.

§ 4 Modification

(Modification 1)

In the above embodiment, an example of the blood pressure data has been described; however, the present invention is not limited thereto. The above embodiment can also be applied to biological data other than the blood pressure data. The biological data may also be data indicating biological information such as an electrocardiogram or a pulse rate. In this case, the medicine prescribed to the patient is directed to a disease that causes abnormalities in the electrocardiogram or pulse rate.

(Other Modifications)

In short, the present invention is not limited to the above-described embodiment and can be embodied in practice by modifying the structural elements without departing from the gist of the invention. Furthermore, various inventions can be formed by appropriately combining a plurality of structural elements disclosed in the embodiment. For example, some of the structural elements may be deleted from the entire set of structural elements shown in the embodiment. Moreover, structural elements described in different embodiments may be suitably combined.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

§ 5 Appendix

A part or all of each of the embodiments described above can be described as shown in the following appendices in addition to the scope of claims, but is not limited thereto.

(Appendix 1)

A data processing apparatus comprising:

a processor configured to:

acquire biological data of a subject;

estimate a remaining amount of a medicine prescribed to the subject;

determine an urgency level regarding the patient's visit to medical personnel based on the biological data and the remaining amount;

generate display data that configures a display screen including a biological data display indicating the biological data, a remaining medicine amount display indicating the remaining amount, and an urgency level display indicating the urgency level; and

output the display data; and

a memory that stores a command to cause the processor to operate.

(Appendix 2)

A data processing apparatus comprising:

a processor configured to:

acquire biological data of a subject;

estimate a remaining amount of a medicine prescribed to the subject;

determine an examination support message for supporting examination of the subject by medical personnel from a plurality of candidates based on the biological data and the remaining amount;

generate display data that configures a display screen including a biological data display indicating the biological data, a remaining medicine amount display indicating the remaining amount, and the examination support message; and

output the display data; and

a memory that stores a command to cause the processor to operate.

(Appendix 3)

A data processing method comprising:

a biological data acquisition process that acquires biological data of a subject using at least one processor;

a remaining amount estimation process that acquires a remaining amount of a medicine prescribed to the subject using at least one processor;

a decision process that determines an urgency level regarding the patient's visit to medical personnel based on the biological data and the remaining amount using at least one processor;

a display data generating process that generates display data that configures a display screen including a biological data display indicating the biological data, a remaining medicine amount display indicating the remaining amount, and an urgency level display indicating the urgency level using at least one processor; and

a display data output process that outputs the display data using at least one processor.

(Appendix 4)

A data processing method comprising:

a biological data acquisition process that acquires biological data of a subject using at least one processor;

a remaining amount estimation process that acquires a remaining amount of a medicine prescribed to the subject using at least one processor;

a decision process that determines an examination support message for supporting examination of the subject by medical personnel from a plurality of candidates based on the biological data and the remaining amount using at least one processor;

a display data generating process that generates display data that configures a display screen including a biological data display indicating the biological data, a remaining medicine amount display indicating the remaining amount, and the examination support message using at least one processor; and

a display data output process that outputs the display data using at least one processor.

### REFERENCE SIGNS LIST

10 . . . Medical personnel display screen server
11 . . . Control unit
12 . . . Storage device
13 . . . Communication I/F
20 . . . Medical personnel portable terminal
30 . . . Sphygmomanometer
40 . . . Weight scale
50 . . . Activity meter
60 . . . Patient portable terminal
70 . . . Data Cloud
80 . . . Data extraction server
111 . . . CPU
112 . . . ROM
113 . . . RAM
201 . . . Search box display
202 . . . Link display
203 . . . Link display
204 . . . Urgency level display
205 . . . Warning display
1111 . . . Biological data acquisition unit
1112 . . . Remaining amount estimation unit
1113 . . . Decision unit
1114 . . . Display data generating unit
1115 . . . Display data output unit

1. An apparatus comprising processing circuitry coupled to a memory, the processing circuitry configured to:
acquire biological data of a subject;
estimate a remaining amount of a medicine prescribed to the subject;
determine an urgency level regarding the subject visit to medical personnel based on the biological data and the remaining amount;
generate display data that configures a display screen including a biological data display indicating the biological data, a remaining medicine amount display indicating the remaining amount, and an urgency level display indicating the urgency level; and
output the display data.

2. The data processing apparatus according to claim 1, wherein the processing circuitry is further configured to generate the display data configuring the display screen including the remaining medicine amount display that varies depending on the remaining amount.

3. The data processing apparatus according to claim 1, wherein the processing circuitry is further configured to estimate the remaining amount based on a date on which the medicine is prescribed, the number of days for which the medicine is prescribed, and information related to medication input by the subject.

4. An apparatus comprising processing circuitry coupled to a memory, the processing circuitry configured to:
acquire biological data of a subject;
estimate a remaining amount of a medicine prescribed to the subject;
determine an examination support message for supporting examination of the subject by medical personnel from a plurality of candidates based on the biological data and the remaining amount;
generate display data that configures a display screen including a biological data display indicating the biological data, a remaining medicine amount display indicating the remaining amount, and the examination support message; and
output the display data.

5. A data processing method comprising:
acquiring biological data of a subject;
acquiring remaining amount of a medicine prescribed to the subject;
determining an urgency level regarding the patient's visit to medical personnel based on the biological data and the remaining amount;
generating display data that configures a display screen including a biological data display indicating the biological data, a remaining medicine amount display indicating the remaining amount, and an urgency level display indicating the urgency level; and
outputting the display data.

6. A non-transitory computer readable medium storing a computer program which is executed by a computer to provide the steps of:
acquiring biological data of a subject;
acquiring remaining amount of a medicine prescribed to the subject;
determining an urgency level regarding the subject visit to medical personnel based on the biological data and the remaining amount;
generating display data that configures a display screen including a biological data display indicating the biological data, a remaining medicine amount display indicating the remaining amount, and an urgency level display indicating the urgency level; and
outputting the display data.

7. A data processing method comprising:
acquiring biological data of a subject;
acquiring remaining amount of a medicine prescribed to the subject;
determining an examination support message for supporting examination of the subject by medical personnel from a plurality of candidates based on the biological data and the remaining amount;
generating display data that configures a display screen including a biological data display indicating the biological data, a remaining medicine amount display indicating the remaining amount, and the examination support message; and
outputting the display data.
